(19) United States
(12) Patent Application Publication
(10) Pub. No.: US 2009/0088725 A1
(43) Pub. Date: Apr. 2, 2009

(54) ACCESSORY SLEEVE FOR A MEDICAL DEVICE

(75) Inventors: Olivier BATAILLE, Chazy, NY (US); Andre TREMBLAY, Laval (CA); Stuart KOZLICK, Cote-St-Luc (CA)

Correspondence Address: CHRISTOPHER & WEISBERG, P.A., 200 EAST LAS OLAS BOULEVARD, SUITE 2040, FORT LAUDERDALE, FL, 33301, US

(73) Assignee: CRYOCATH TECHNOLOGIES INC., Kirkland (CA)

(21) Appl. No.: 11/862,264

(22) Filed: Sep. 27, 2007

(51) Int. Cl. A61M 25/01; A61M 25/10

(52) U.S. Cl. 604/509; 604/264

(57) ABSTRACT

The present invention provides a medical system including a protective sleeve for a medical device having a distal end positionable in a blood vessel, the protective sleeve including a body defining a passage therethrough and a vent in fluid communication with the passage, where at least a portion of the distal end of the medical device is positionable within the passage. An introducer device may be included having a sheath, where the sheath defines a first passage for receiving a portion of the catheter. In addition, the sleeve may be releasably engageable with the introducer device for subsequent insertion of the medical device into the introducer device.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a medical device system and method of use thereof, and in particular, towards an accessory for an intravascular device.

### BACKGROUND OF THE INVENTION

Balloon catheters are used in a variety of medical procedures, such as angioplasty, ablation, and dilation procedures. Balloon catheters typically are used in arteries that are narrow, such as the coronary arteries, and in circumstances where the naturally narrow artery is even further narrowed by a stenotic obstruction. In such circumstances, it is essential for the balloon to have a low profile in order to perform the procedure, i.e., that the balloon or distal end of the catheter have a small, effective deflated cross-sectional diameter so that it can be advanced into the particular vessel. To that end, the balloon of an intravascular catheter is typically formed from a very thin polymeric material that can be wrapped or otherwise situated closely about the shaft of the catheter to minimize the profile. The core or inner tube diameter of the catheter should be minimized along with the balloon, which can be done by folding, wrapping or twisting the balloon to achieve the smallest profile possible or by reducing wall thicknesses, to the extent possible, of the balloon itself. Although an undamaged balloon is capable of developing high pressures under inflation, the balloons are delicate and may be susceptible to minor scratches or other damage during handling that could result in premature balloon failure. As a result, it is a common practice to enclose the balloon portion of the catheter in a sleeve-like balloon protector that remains in place until the catheter is prepared for use by the physician. A protective sleeve may also maintain the balloon in its tightly wrapped, low profile configuration during shipment and storage.

Moreover, with certain balloon materials, such as polyolefin, a sterilization process involving the exposure of the catheter and/or balloon to high temperature causes the balloon to be “heat set” in the folded or wrapped condition in which it is held by the protective sleeve. As a result, when the balloon protector is later removed, the balloon remains in a tightly wrapped condition. This heat set of the balloon has a further advantage in that when the balloon is inflated and is then deflated, the application of a negative fluid pressure during deflation will cause the balloon to tend to return to its previous heat set, tightly wrapped shape. This greatly facilitates the removal of the catheter after the dilatation procedure has been performed.

In any given intravascular procedure, a balloon catheter is typically inserted into the femoral artery through an insertion sheath, and moved up towards the heart and/or other accessible area of interest. A passageway for inflating and deflating the balloon extends through the catheter and is connected at its proximal end to an external pump, where the proximal end of the catheter remains outside of the patient's body. The insertion sheath generally has a hemostatic valve at one end to prevent blood from traveling up the annular space between the catheter and the insertion sheath and escaping from the proximal end of the insertion sheath. The hemostatic valve may include a hub for connection with a protective sleeve, where the sleeve is disposed about the catheter and may be secured to the hub. However, in coupling the protective sleeve of the catheter to the hub or distal portion of the introducer sheath/hemostatic valve, fluid may be forced into the valve and/or create a siphoning effect upon sealing the sleeve with the valve. In addition, a protective sleeve for a particular medical device may not readily engage with and/or otherwise couple to a hub and/or hemostatic valve of an introducer sheath. In light of the above, it would be desirable to provide a protective sleeve for a medical device that is readily engageable with various introducer sheaths and/or hemostatic valve devices, and to further reduce the likelihood of introducing and/or siphoning fluids upon connection of the sleeve to the sheath.

### SUMMARY OF THE INVENTION

The present invention advantageously provides medical system and method of use including a protective sleeve for a medical device that is readily engageable with various introducer sheaths and/or hemostatic valve devices, and to further reduce the likelihood of introducing and/or siphoning fluids upon connection of the sleeve to the sheath. In particular, the present invention may include a protective sleeve for a medical device having a distal end positionable in a blood vessel, where the protective sleeve includes a body defining a first opening, a second opening, and a passage extending from the first opening to the second opening such that at least a portion of the distal end of the medical device is positionable within the passage. The sleeve body may define a bezel-shaped or angled tip proximate the first opening. The sleeve body may further define a vent in fluid communication with the passage. For example, the sleeve body may define a lip proximate the first opening, and the vent may include an opening in the lip. The sleeve may have a substantially rigid construction to prevent the destruction or deformation of the medical device, and to further provide a stable component through which the medical device may be positioned. The sleeve may be substantially transparent for viewing any fluid or substance therein. In addition, the sleeve may also include a port in fluid communication with the passage, where the port may include a protrusion extending from the body, with the protrusion defining a channel in fluid communication with the passage.

The present invention may include a medical device having an elongate body defining a proximal end, a distal end, and a fluid flow path therethrough. An inflatable element, such as one or more balloons or the like, may be coupled to the distal end of the elongate body in fluid communication with the fluid flow path. In addition, a sleeve may be provided defining a first opening, a second opening, and a passage extending from the first opening to the second opening, where the sleeve defines a vent in fluid communication with the passage, and where at least a portion of the inflatable element is positionable within the passage. The sleeve may define a lip proximate the first opening such that the vent is an opening in the lip.

The present invention may include a medical device for use with a catheter, including an introducer device having a sheath, where the sheath defines a first passage for receiving a portion of the catheter. The introducer device may further include a valve disposed within the first passage. Moreover, a sleeve may be provided defining a first opening, a second opening, and a second passage extending from the first opening to the second opening. The sleeve may define a vent in fluid communication with the passage, and at least a portion of the catheter may be positionable within the passage. The sleeve may further be releasably engageable with the introducer device to place the second passage in fluid communication with the first passage.

A method provided by the present invention may include the steps of providing a medical device having an elongate body defining a proximal end, a distal end, and a fluid flow path therethrough; providing a sleeve defining a first opening, a second opening, and a first passage extending from the first opening to the second opening; providing an introducer device defining a second passage; positioning the distal end of the medical device in the first passage of the sleeve; releasably engaging the sleeve with the introducer device such that the first and second passages are in fluid communication; and providing a fluid flow path from at least one of the first and second passages to atmosphere.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates an embodiment of a medical device system in accordance with the present invention;

FIG. 2 is a perspective view of an embodiment of an accessory sleeve for a medical device in accordance with the present invention;

FIG. 3 depicts a cross-sectional view of the accessory sleeve of FIG. 2;

FIG. 4 is a cross-sectional view of an embodiment of an accessory sleeve for a medical device in accordance with the present invention;

FIG. 5 shows a front view of the accessory sleeve of FIG. 2;

FIG. 6 is a cross-sectional view of an embodiment of an accessory sleeve coupled to an introducer device in accordance with the present invention; and

FIG. 7 is an additional cross-sectional view of an embodiment of an accessory sleeve coupled to an introducer device in accordance with the present invention.

### DETAILED DESCRIPTION OF THE INVENTION

The present invention provides a medical system including an accessory sleeve for use with a medical device, where the protective sleeve may be movably positionable about a portion of the medical device. The sleeve may further be engageable with an introducer device or the like to facilitate coupling of the medical device to the introducer device for a particular medical procedure, while preventing the undesired introduction of fluid into the introducer device and/or to prevent an undesired pressure build-up between the devices. Details of the various components of the present invention are provided below and in the accompanying figures, in which like reference designators refer to like elements.

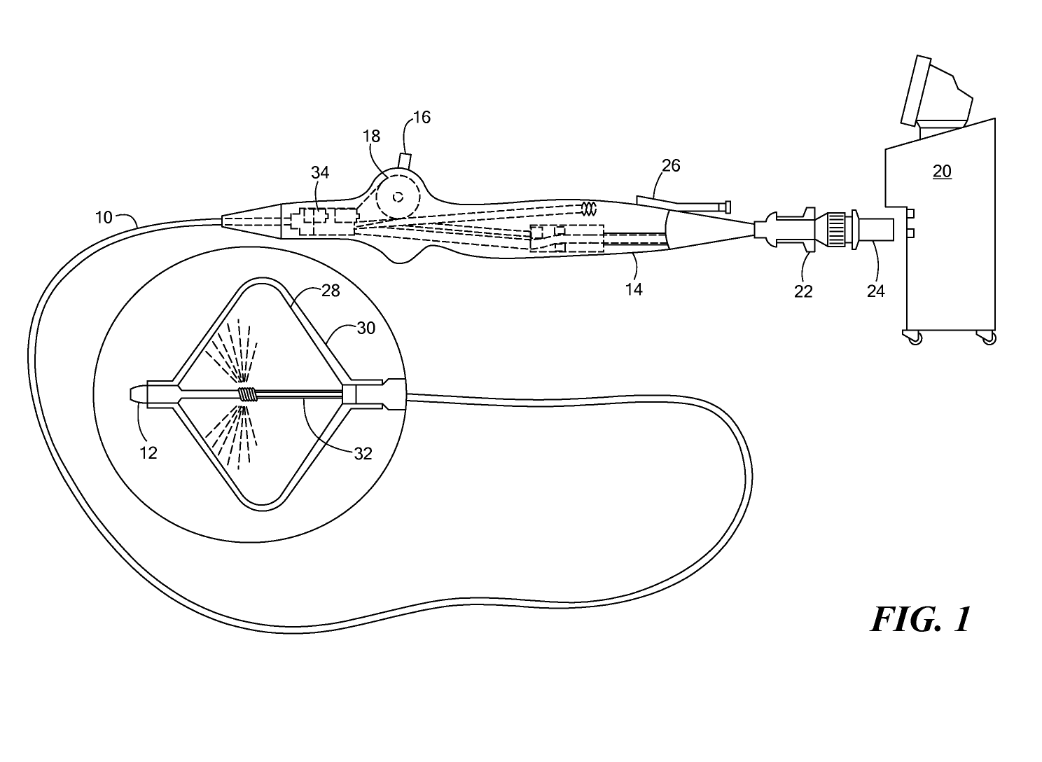

Now referring to FIG. 1, a medical device system is illustrated. The system includes an elongate, highly flexible catheter 10 that is suitable for passage through the vasculature. The catheter includes a catheter body having a distal end 12 positionable within the vasculature of a patient. The distal end 12 is shown magnified and are described in greater detail below. The catheter body has a proximal end that is mated to a handle 14 that can include an element such as a lever or knob 16 for manipulating the catheter body and the distal end 12. In an exemplary embodiment, a pull wire with a proximal end and a distal end has its distal end anchored to the catheter at or near the distal end. The proximal end of the pull wire is anchored to an element such as a cam 18 in communication with and responsive to the lever 14. The handle 14 can further include circuitry for identification and/or use in controlling of the catheter or another component of the system.

Continuing to refer to FIG. 1, the handle 14 can also include connectors that are matable directly to a control unit 20 or indirectly by way of one or more umbilicals. In the system illustrated, the handle 14 is provided with a first connector 22 that is matable with a co-axial fluid umbilical (not shown) and a second connector 24 that is matable with an electrical umbilical (not shown) that can further include an accessory box (not shown). In the exemplary system, a fluid supply and exhaust, as well as various control mechanisms for the system may be housed in a single console. The handle may also be provided with a fitting 26 for receiving a guide wire (not shown) that is passed into a guide wire lumen.

Still referring to FIG. 1, the distal end 12 of the catheter 10 is shown as including one or more inflatable or otherwise expandable elements. For example, the distal end 12 may include a double balloon having a first membrane (e.g., inner balloon) 28 contained or enclosed within a second membrane (e.g., outer balloon) 30, thereby defining an interface or junction between the first and second membranes. The second membrane 30 may provide a safeguard to prevent fluid from leaking out of the device and into surrounding tissue should the first membrane 28 rupture or develop a leak. A fluid supply tube 32 in fluid communication with a coolant supply in the control unit 20 may be provided to release fluid from one or more openings in the tube within the inner balloon in response to commands and other control input.

The handle 14 of the medical device may include one or more sensors 34 to monitor the various operating parameters of the device, such as fluid pressure, temperature, flow rate, and the like within one or both of the balloons, as well as to detect blood ingress.

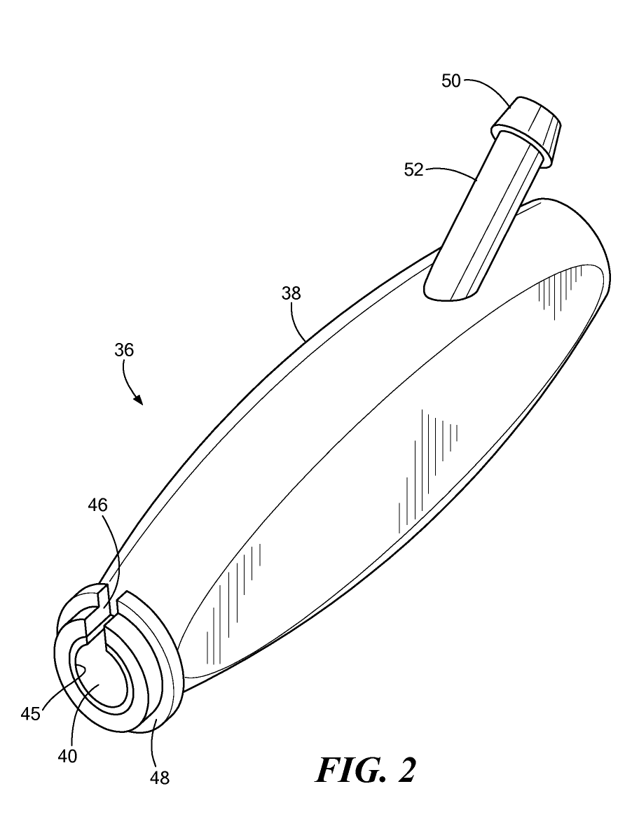

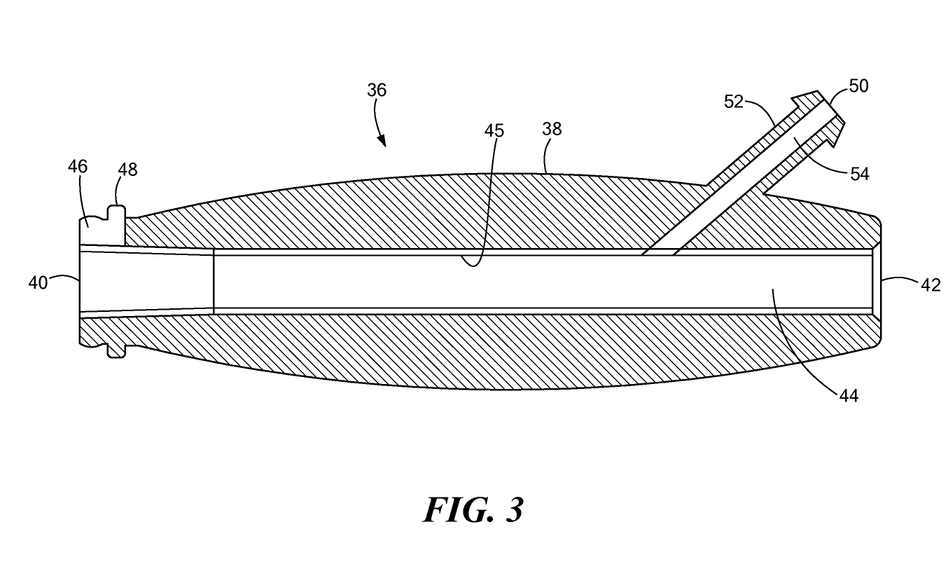

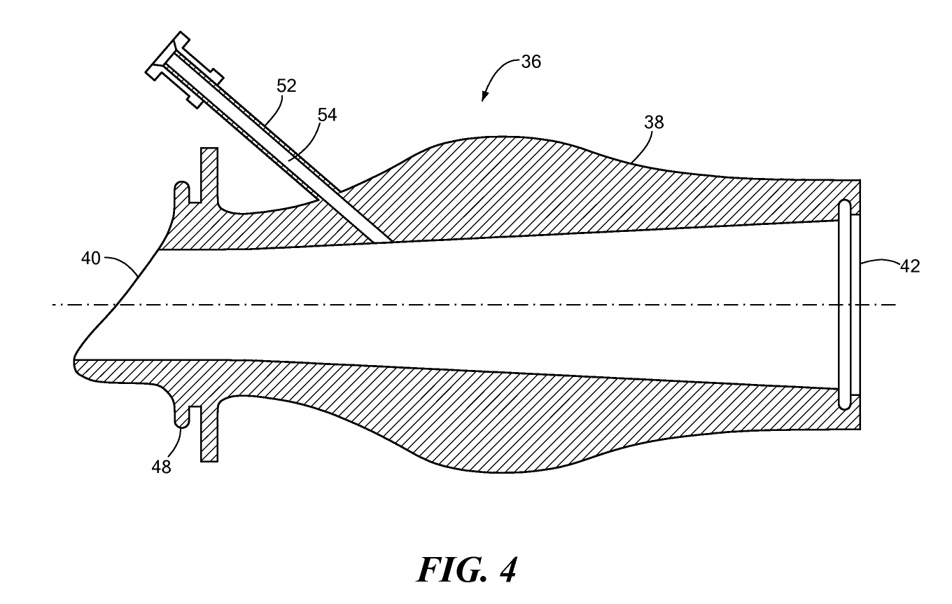

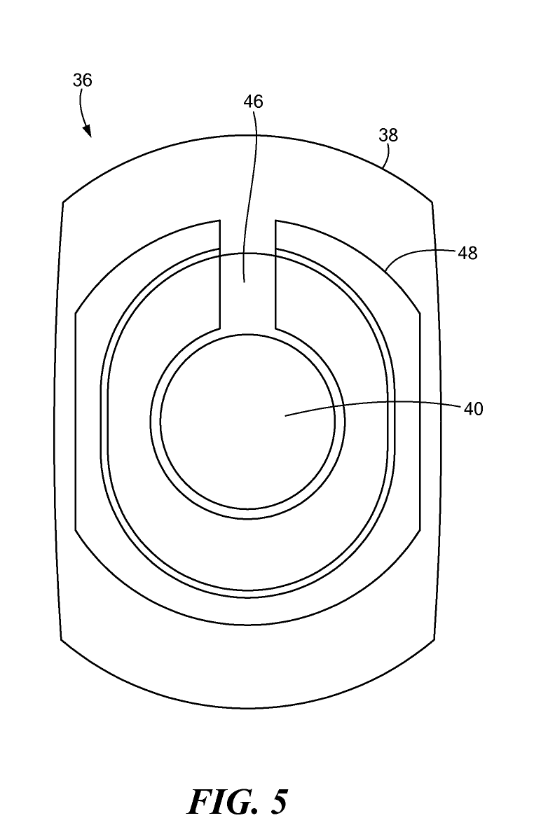

Now referring to FIGS. 2-5, the present invention may include a sleeve 36 movably positionable about a portion of the medical device described above. For example, the sleeve 36 may be positionable about the distal portion 12 of the catheter 10 and/or a portion of the balloons. The sleeve 36 may generally include a body 38, where the body includes a first opening 40, a second opening 42, and a passage 44 extending from the first opening 40 to the second opening 42. Either and/or both of the first and second openings may include a beveled or tapered region to ease and facilitate insertion of a portion of the medical device or catheter 10 into the passage. The sleeve 36 body may be generally contoured, and may further include one or more flattened surfaces to aid in grasping and handling the sleeve 36. Further, the sleeve 36 may include a contoured or angled bezel profile proximate to or surrounding the first opening 40, as shown in FIG. 4. The angled profile may aid in the insertion of a portion of the sleeve into a complementary receiving end of a medical device. The sleeve 36 may generally be constructed from a rigid or otherwise durable material to prevent the distal end 12 of the medical device 10 from being damaged and to further decrease the likelihood that the sleeve 36 is damaged or deformed prior to use. Moreover, the sleeve may be substantially transparent to provide visual confirmation of the presence and/or absence of fluid or other substances in the sleeve at any point during a procedure, such as blood, coolant, or the like.

The passage 44 of the sleeve 36 may accommodate a portion of the medical device, such as the one or more balloons for example. In addition, the passage 44 may include a plurality of diameters throughout its length. For example, a portion of the passage 44 leading to the first opening 40 may have a larger diameter than a portion of the passage 44 proximate the second opening 42. Alternatively, the opening 44 may be tapered and include a diameter that increases from the first opening 40 to the second opening 42, as shown in FIG. 4. The walls of the passage 44 may include an internal surface finish or layer 45 of a low-friction material to aid in the passage of a portion of the medical device through the passage. For example, the interior of the passage 44 may be coated or molded with a layer of polytetrafluoroethylene (PTFE). Similarly, a tube or insert constructed from a material having a coefficient of friction less than that of the passage 44 itself may be inserted within the passage prior to use of the sleeve 36.

The sleeve 36 may further define a vent 46 in fluid communication with the passage 44 to allow fluid to escape and/or be entrained into the passage 44 when the sleeve 36 is engaged with or otherwise coupled to a hemostatic valve or the like during a medical procedure. The vent 46 may be disposed proximate the first opening 40 in the body of the sleeve 36. In particular, the sleeve 36 body may define a ridge or lip 48 that circumscribes or otherwise outlines a portion of a perimeter of the first opening 40. The vent 46 may include an opening, aperture, or depression in the lip or ridge that allows fluid communication between the passage 44 and the exterior atmosphere even when the first opening 40 and/or ridge 48 abuts or otherwise connects to a secondary apparatus.

The sleeve 36 may further include a port 50 to allow for the flushing or removal of excess fluid prior to use of the sleeve 36 in a procedure. The port 50 may include an opening in the body 38 in fluid communication with the passage 44, and further, the port 50 may include a protrusion 52 extending from the sleeve 36 body, where the protrusion 52 defines a channel 54 in fluid communication with the passage 44 and the exterior environment. The protrusion 52 may be angled towards the rear and/or second opening of the sleeve body, and further, the protrusion may include a flared end to aid in engaging a tube, hose or other element to facilitate the introduction and/or removal of fluid to and from the sleeve 36. Alternatively, the port 50 may be angled towards the front of the sleeve 36, as shown in FIG. 4, and the port may include a pliable or bendable hose or conduit to allow for the manipulation of the positioning of the port 50 when the sleeve 36 is in use.

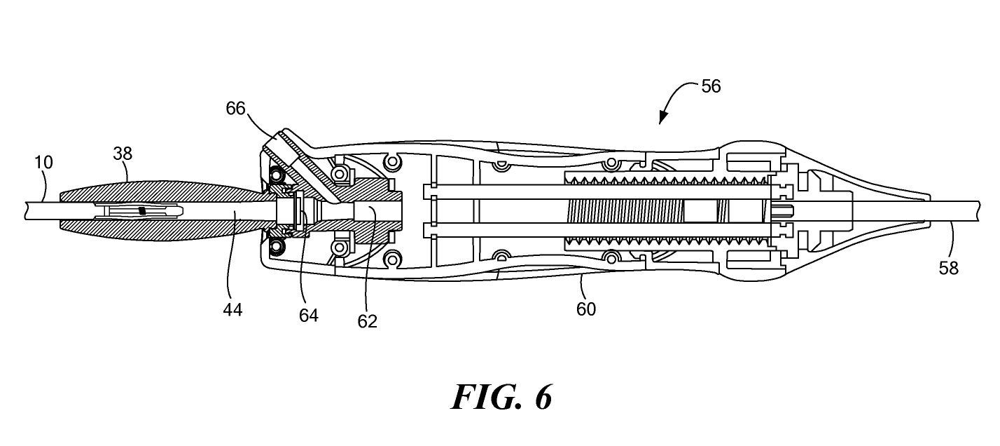

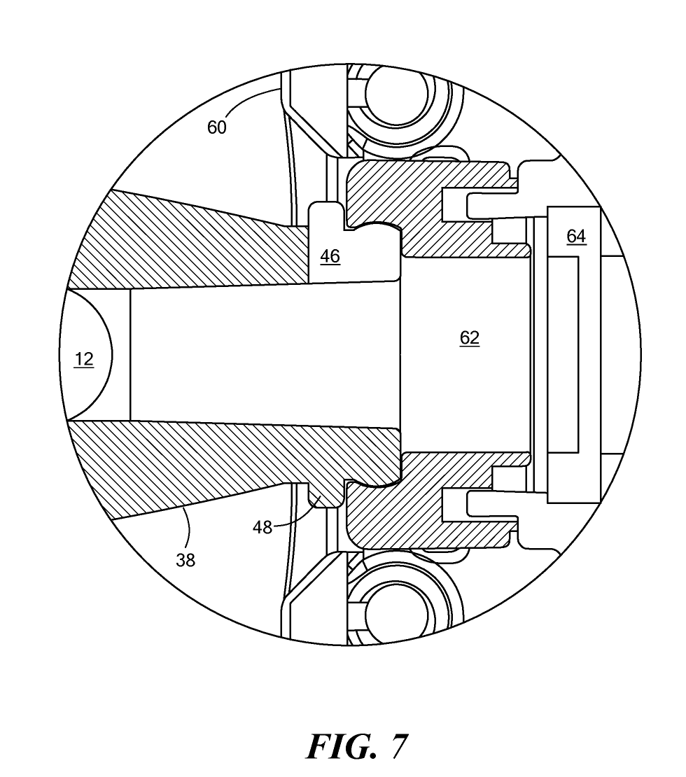

Now referring to FIGS. 6 and 7, the present invention may further include an introducer device 56 used in conjunction with an intravascular device for accessing and/or otherwise positioning a portion of the medical device 10 within the body. The introducer device 56 may generally include an introducer sheath or an insertion sheath 58 that is inserted into or otherwise positioned about an entry into the body. The introducer device 56 provides a passage or entry point into the body through which the medical device may be inserted. In a particular example, the introducer device 56 may include a sheath 58 positionable within the vasculature of a patient. The sheath 58 may be flexible, and may define a path therethrough for receiving at least a portion of the medical device. The introducer device 56 may further include a handle 60 coupled to a proximal end of the sheath 58. The handle 60 may include a steering mechanism to manipulate the movement and/or configuration of the sheath 58. The handle 60 may further define an insertion passage 62 extending through and in fluid communication with the sheath 58. The insertion passage 62 may extend through the complete length of the handle 60 from an insertion opening at one end of the handle 60 to the sheath 58 at the opposite end of the handle 60, thereby allowing a portion of the medical device 10 to pass through the handle portion 60 and into the sheath 58.

The introducer device 56 may further include a valve 64 disposed within the insertion passage 62 of the handle 60 and/or portion of the sheath 58. The valve 64 may reduce and/or eliminate the uncontrolled entry or release of fluids to and from the introducer device 56. The valve 64 may include a check valve, a hemostasis valve, or the like for control of fluid movement. The introducer device 56 may further include an introducer port 66 in fluid communication with the insertion passage 62 and/or the sheath 58 of the device. The introducer port 66 can provide an auxiliary entryway for the injection and/or removal of fluids to and from the introducer device 56.

The introducer device 56 may be removably engageable with the sleeve 36 of the present invention, and may further provide a fluid flow path from the insertion passage 62 and/or passage 44 of the sleeve 36 to the surrounding atmosphere. For example, at least a portion of the sleeve 36 body may be matable with the insertion opening of the handle 60 of the introducer device 56. In particular, the first opening 40 of the sleeve 36 may be positionable within a portion of the insertion passage 62 of the introducer device 56, such that a portion of the lip 48 abuts or otherwise engages the handle portion 60. The sleeve 36 may form a frictional engagement and/or substantial seal with the handle 60 of the introducer device 56. Moreover, the vent 46 of the sleeve 36 may extend through the lip 48, and may further provide for fluid communication between a portion of the insertion passage 62 and the surrounding environment upon engagement of the sleeve 36 to the introducer device 56.

In an exemplary use of the present invention, the medical device 10 may be prepared for insertion into the passage 44 of the sleeve 36, and may include folding and/or flattening the balloon(s) on the distal end 12 of the medical device 10. Upon such steps, the medical device 10 may be slidably positioned within the passage 44 of the sleeve 36 to substantially cover and/or protect the balloons of the device. During a subsequent medical procedure in which the medical device may be employed, at least a portion of the introducer device 56 may first be positioned or otherwise inserted into the vasculature or tissue of a patient. The introducer device 56 may be configured such that a portion of the sheath 58 is inserted within the patient, while the handle 60 at the proximal end of the sheath 58 extends out of the body and is available to receive a portion of the sleeve 36 and/or medical device 10. The sleeve 36, which is positioned about the distal end 12 of the medical device 10, may be directed towards the proximal end of the introducer device 56. In particular, the sleeve 36 may be releasably engaged with the insertion opening of the handle 60 of the introducer device 56, thereby placing the passage 44 of the sleeve 36 and the insertion passage 62 of the introducer device 56 in fluid communication with one another.

The sleeve 36 may engage the introducer device 56 through a frictional fit or other suitable releasable engagement mechanisms as known in the art. The engagement of the sleeve 36 to the introducer device 56 may include positioning the lip 48 of the sleeve 36 such that it abuts a portion of the handle 60 and/or insertion passage 62, while the vent 46 provides a fluid path from the insertion passage 62 to the surrounding atmosphere. Accordingly, the passage 44 of the sleeve 36 and the insertion passage 62 of the introducer device 56 are substantially aligned, and the sleeve 36 provides a substantially rigid guide for the movement of a portion of the medical device 10 into the introducer device 56. As the medical device 10 is moved, any excess air or fluid which would otherwise be forced into the introducer element 56 and thus the sheath 58 by the introduction of the medical device passes through the vent 46 to prevent the undesired introduction or siphoning of fluid into and/or out of the introducer device 56 and the patient. Of note, although the vent 46 is shown and described as a feature and/or component on the sleeve 36 body, it is contemplated that a vent 46 providing for a similar release of undesired fluid may be included on a portion of the introducer device 56 as well. In an embodiment where a port 50 is provided on the sleeve 36 body, the port 50 may be coupled to a syringe or other fluid source to direct a fluid, such as saline, into the passage 44 and into the introducer device 56 to aid in the release of excess air or fluid. Once the sleeve 36 and/or introducer device 56 have been appropriately flushed, the medical device lo may then continue to be inserted through the length of the introducer device 56, the valve 64, and into the sheath 58 to perform a particular procedure or treatment.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An accessory sleeve for a medical device having a distal end positionable in a blood vessel, the accessory sleeve comprising:
a body defining a first opening, a second opening, and a passage extending from the first opening to the second opening, wherein the body defines a vent in fluid communication with the passage, wherein the body is substantially rigid, and wherein at least a portion of the distal end of the medical device is positionable within the passage.

2. The accessory sleeve according to claim 1, wherein the body defines a lip proximate the first opening, and wherein the vent is an opening in the lip.

3. (canceled)

4. The accessory sleeve according to claim 1, wherein the body defines a bezel tip proximate the first opening.

5. The accessory sleeve according to claim 1, further comprising a port in fluid communication with the passage.

6. The accessory sleeve according to claim 5, wherein the port includes a protrusion extending from the body, the protrusion defining a channel in fluid communication with the passage.

7. The accessory sleeve according to claim 1, further comprising a layer of material having a low coefficient of friction disposed about at least a portion of the passage.

8. The accessory sleeve according to claim 1, wherein the body is substantially transparent.

9. A medical device, comprising:
an elongate body defining a proximal end, a distal end, and a fluid flow path therethrough;
an inflatable element coupled to the distal end of the elongate body in fluid communication with the fluid flow path; and
a sleeve defining a first opening, a second opening, and a passage extending from the first opening to the second opening, wherein the sleeve defines a vent in fluid communication with the passage, wherein the sleeve is substantially rigid, and wherein at least a portion of the inflatable element is positionable within the passage.

10. The medical device according to claim 9, wherein the sleeve defines a lip proximate the first opening, and wherein the vent is an opening in the lip.

11. (canceled)

12. The medical device according to claim 9, wherein the sleeve defines a port in fluid communication with the passage.

13. The medical device according to claim 12, wherein the port includes a protrusion extending from the sleeve, the protrusion defining a channel in fluid communication with the passage.

14. A medical device for use with a catheter, comprising:
an introducer device having a sheath, wherein the sheath defines a first passage for receiving a portion of the catheter; and
a substantially rigid sleeve defining a first opening, a second opening, and a second passage extending from the first opening to the second opening, wherein the sleeve defines a vent in fluid communication with the passage, wherein at least a portion of the catheter is positionable within the passage, and wherein the sleeve is releasably engageable with the introducer device to place the second passage in fluid communication with the first passage.

15. The medical device according to claim 14, wherein the introducer device includes a valve disposed within the first passage.

16. The medical device according to claim 14, wherein the sleeve defines a lip proximate the first opening, and wherein the vent is an opening in the lip.

17. (canceled)

18. The medical device according to claim 14, wherein the sleeve defines a port in fluid communication with the passage.

19. The medical device according to claim 18, wherein the port includes a protrusion extending from the sleeve, the protrusion defining a channel in fluid communication with the passage.

20. The medical device according to claim 13, wherein a substantial portion of the second passage is concentric with a portion of the first passage upon releasable engagement of the sleeve to the introducer device.

21. A method of performing a medical procedure, comprising the steps of:
slidably positioning a portion of a medical device into a passage of a substantially rigid sleeve;
releasably engaging the sleeve with an introducer device, where the releasable engagement between the sleeve and the introducer element defines a vent; and
slidably inserting a portion of the medical device into the introducer device.

22. The method according to claim 21, wherein the medical device is a catheter having one or more inflatable elements disposed thereon.

23. The method according to claim 21, wherein the introducer device includes a sheath able to receive a portion of the medical device therein.

24. The method according to claim 21, wherein the step of releasably engaging the sleeve with the introducer device includes positioning a substantial portion of the passage concentric to an opening of the introducer device.
